(19) United States
(12) Patent Application Publication
(10) Pub. No.: US 2008/0039527 A1
(43) Pub. Date: Feb. 14, 2008

(54) Calixarenes as Inhibitors of Protein Kinase B

(75) Inventors: Rudiger Woscholski, London (GB); Helen Claire Hailes, Kent (GB); Macba Golda Numbere, London (GB)

Correspondence Address: ELMORE PATENT LAW GROUP, PC, 209 MAIN STREET, N. CHELMSFORD, MA, 01863, US

(73) Assignee: Imperial College Innovations Ltd.

(21) Appl. No.: 10/570,860

(22) PCT Filed: Sep. 9, 2004
(86) PCT No.: PCT/GB04/03858

§ 371 Date: Apr. 12, 2007

(30) Foreign Application Priority Data
Sep. 9, 2003 (GB) 0321089.5

(51) Int. Cl. A61K 31/136; A61K 31/195; A61P 25/28; C07C 211/01; C07C 61/08

(52) U.S. Cl. 514/567; 514/649; 562/507; 564/372

(57) ABSTRACT

The present invention provides compounds useful in inhibiting protein kinase B (PKB/Akt). Compositions comprising such compounds and their use are also provided.

The present invention relates to novel compounds, which are useful as inhibitors and/or activators of protein kinase B (PKB/Akt). As such, these compounds will be useful in the treatment of cancer

Phosphoinositide 3-kinases (PI 3-kinase) are an evolutionary conserved family of enzymes possessing lipid kinase activity who in response to extracellular stimuli are capable of generating a series of 3-phosphorylated phosphoinositide lipids with signalling potential. The resulting cellular effects of PI 3-kinase activity are diverse, including DNA synthesis, chemotaxis, glucose transport and vesicle trafficking. The activation of PI 3-kinases themselves takes place via a number of mechanisms, including receptor tyrosine kinases, Ras and heterotrimeric G-proteins.

One effector of PI 3-kinase responsible for some of the aforementioned effects is protein kinase B (PKB/Akt), a mammalian homologue of the viral oncoprotein v-akt (Staal 1987). PKB is recruited to the plasma membrane in response to growth factor stimulation via the binding of 3-phosphoinositides to its PH domain which facilitates its phosphorylation at two distinct sites and subsequent activation. The first phosphorylation site, threonine-308 (T308) lies in the activation loop of PKB and is phosphorylated by phosphoinositide-dependent kinase-1 (PDK-1). The second site, serine-473 (S473) lies in the c-terminal hydrophobic regulatory domain, and is phosphorylated by an as yet unidentified kinase(Chang, Lee et al. 2003). To date several S473 candidate kinases have been postulated, including PDK-1, mitogen-activated protein kinase-activated protein kinase 2, intergrin-linked kinase (ILK) and PKB itself (Brazil, Park et al. 2002; Hill, Feng et al. 2002). It remains to be seen whether any of these kinases or a so far unidentified kinase is responsible for the phosphorylation of this particular site. Other protein kinases of the AGC kinase family such as protein kinase C delta (PKCδ) and p70^{S6K} share a similar activation mechanism via the phosphorylation of their homologous residues (Newton 2003). The activation of all the aforementioned kinases is susceptible to PI 3-kinase inhibition by LY294002 and wortmannin. Effectors of PKB include Bad, GSK-3 (glycogen synthase kinase-3) and mTOR (mammalian target of rapamycin)(Vivanco and Sawyers 2002). mTOR is a regulator of protein synthesis and is instrumental in PKCδ activation (Parekh, Ziegler et al. 2000). Like PI 3-kinase, studies of mTOR signalling have been aided by the use of pharmacological agents. mTOR activity is inhibited by rapamycin, via its binding to FKBP12, thus inhibiting events distal to mTOR (Sabers, Martin et al. 1995).

Thus, Phosphoinositide signalling is a key element in controlling cell death, survival and fate. In particular, cell survival is an important mechanism of the natural defence against cancer. Cell survival is controlled by phosphoinositide 3-kinase products, which in turn activate a particular protein kinase, called PKB or Akt. PKB/Akt is phosphorylated by other kinases subsequently leading towards full activation of its own catalytic abilities and thus progressing the cell survival signal through this protein kinase cascade. Unravelling the elements in control of PKB phosphorylation has been the focus of many research groups and drug development teams.

We have now identified compounds which are capable of inhibiting and/or activating PKB.

Thus, in a first aspect, the present invention provides a compound of the formula:

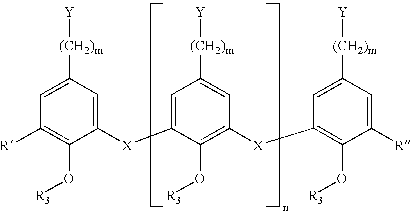

wherein Y is H, halogen, OH, CO₂H, CONR₁R₂, CHO or NR₁R₂, R₁, R₂ and R₃ are each independently H or C₁₋₅ alkyl;

n is 2-10 and m is 1-5;

R′ and R″ are independently H or CH₂OH;

X is CH₂, O, CH₂O or CH₂OCH₂,

and pharmaceutically acceptable salts thereof.

Compounds where n is 2, 3, 4, 5, 6, 7, 8, 9 or 10 are provided and each forms a separate embodiment of the invention.

Preferred compounds for use as inhibitors of PKB are compounds of the formula:

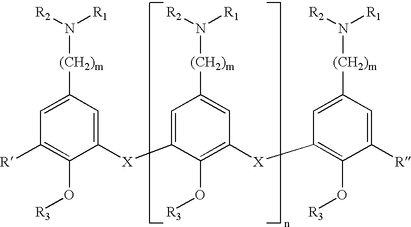

wherein R₁, R₂ and R₃ are each independently H or C₁₋₅alkyl, n is 2-10 and m is 1-5;

R′ and R″ are independently H or CH₂OH and X is CH₂, O, CH₂O or CH₂OCH₂,

and pharmaceutically acceptable salts thereof.

Particularly preferred compounds are those where R₃ is Me and Y is NHMe.

In one embodiment of the invention, compounds which activate PKB are those where Y is halogen. In the context of the present invention, halogen means F, Cl, I or Br, preferably Cl, I or Br.

In a second aspect, the invention provides a compound of the formula:

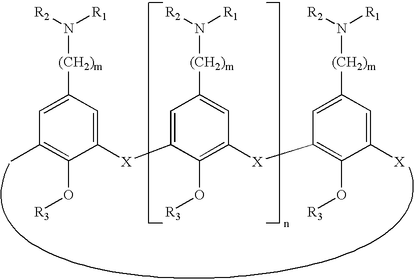

R₁, R₂ and R₃ are each independently H or C₁₋₅alkyl;

n is 1-10 and m is 1-5;

X is CH₂, O, CH₂O or CH₂OCH₂,

and pharmaceutically acceptable salts thereof.

Thus, compounds where n is 1, 2, 3, 4, 5, 6, 7, 8, 9 or 10 are provided and each forms a separate embodiment of this aspect of the invention.

In a third aspect the present invention provides a compound of the formula:

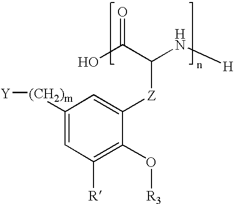

wherein Y is H, halogen, OH, CO₂H, CONR₁R₂, CHO or NR₁R₂, R₁, R₂ and R₃ are each independently H or C₁₋₅alkyl;

n is 2-12 and m is 1-5;

R′ is H or CH₂OH;

Z is a spacer such as (CH₂)ₙ or PEG;

and pharmaceutically acceptable salts thereof.

As discussed herein, the compounds of the present invention find use as inhibitors and/or activators of of PKB, and thus as agents for use in the treatment of cancer. In particular, the compounds described herein find use in cancers where up regulation of PKB is implicated and more particularly where up-regulation together with mutation of PTEN is implicated. Thus, cancers such as ovarian, breast, prostrate, thyroid and pancreatic cancers are particular targets of the compounds.

Those compounds described herein as activators find use in preventing cell death. Thus, they find use in treating degenerative disorders degenerative diseases of those tissues that are unable to reproduce, ie neurons (Alzheimer, stroke, etc) or heart (infarct, hypoxia) and skeletal muscle (sports injuries) tissue, respectively (Glass 2003; Matsui, Nagoshi et al. 2003; Tatton, Chen et al. 2003).

Thus, in a fourth aspect, the present invention provides a pharmaceutical composition comprising at least one compound of the invention, optionally together with one or more pharmaceutically acceptable excipients, diluents or carriers.

The compositions of the invention may be presented in unit dose forms containing a predetermined amount of each active ingredient per dose. Such a unit may be adapted to provide 5-100 mg/day of the compound, preferably either 5-15 mg/day, 10-30 mg/day, 25-50 mg/day 40-80 mg/day or 60-100 mg/day. For compounds of formula I, doses in the range 100-1000 mg/day are provided, preferably either 100-400 mg/day, 300-600 mg/day or 500-1000 mg/day. Such doses can be provided in a single dose or as a number of discrete doses. The ultimate dose will of course depend on the condition being treated, the route of administration and the age, weight and condition of the patient and will be at the doctor's discretion.

The subject of the present invention is most preferably administered in the form of appropriate compositions. As appropriate compositions there may be cited all compositions usually employed for systemically or locally administering drugs. The pharmaceutically acceptable carrier should be substantially inert, so as not to act with the active component. Suitable inert carriers include water, alcohol, polyethylene glycol, mineral oil or petroleum gel, propylene glycol and the like. Said pharmaceutical preparations may be formulated for administration in any convenient way for use in human or veterinary medicine.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam.

However, in certain embodiments the subject agents may be simply dissolved or suspended in sterile water. In certain embodiments, the pharmaceutical preparation is non-pyrogenic, i.e., does not elevate the body temperature of a patient. The phrase “effective amount” as used herein means that amount of one or more agent, material, or composition comprising one or more agents of the present invention which is effective for producing some desired effect in an animal. It is recognized that when an agent is being used to achieve a therapeutic effect, the actual dose which comprises the “effective amount” will vary depending on a number of conditions including the particular condition being treated, the severity of the disease, the size and health of the patient, the route of administration, etc. A skilled medical practitioner can readily determine the appropriate dose using methods well known in the medical arts. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, one or more agents may contain a basic functional group, such as amino or alkylamino, and are, thus, capable of forming pharmaceutically acceptable salts with pharmaceutically acceptable acids.

The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (Berge, Bighley et al. 1977). The pharmaceutically acceptable salts of the agents include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2acetoxybenzoic, fumaric, toluenesulfonic, methanesulfonic, ethane disulfonic, oxalic, isothionic, and the like. In other cases, the one or more agents may contain one or more acidic functional groups and, thus, are capable of forming pharmaceutically acceptable salts with pharmaceutically acceptable bases. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine.

Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (see, for example, Berge et al., supra) Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Formulations of the present invention include those suitable for oral, nasal, topical (including buccal and ublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 percent to about ninety-nine percent of active ingredient, preferably from about 5 percent to about 70 percent, most preferably from about 10 percent to about 30 percent. Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each ontaining a predetermined amount of a compound of the present invention as an active ingredient. A compound of the present invention may also be administered as a bolus, electuary or paste. In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, olyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin apsules using such excipients as lactose or milk sugars, as well as high molecular weight polyethylene glycols and the like. A tablet may be made by compression or molding, optionally with one or more accessory ingredients. Compressed tablets may be repared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients. Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the agents. Formulations of the present invention which are suitable for vaginal administration also include pessaries, tampons, creams, gels, pastes, foams or spray formulations containing such carriers as are known in the art to be appropriate. Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery of a compound of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin. In some cases, in order to prolong the effect of an agent, it is desirable to slow the absorption of the agent from subcutaneous or ntramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle. Injectable depot forms are made by forming microencapsule matrices of the subject compounds in biodegradable polymers such as polylactide-polyglycolide. Depending on the ratio of agent to polymer, and the nature of the particular polymer employed, the rate of agent release can be controlled. Examples of other biodegradable polymers include poly(orthoesters) and poly(anhydrides). Depot injectable formulations are also prepared by entrapping the agent in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier. Apart from the above-described compositions, use may be made of covers, e.g., plasters, bandages, dressings, gauze pads and the like, containing an appropriate amount of a therapeutic. As described in detail above, therapeutic compositions may be administered/ delivered on stents, devices, prosthetics, and implants.

Compounds of the invention can be prepared according to the following scheme:

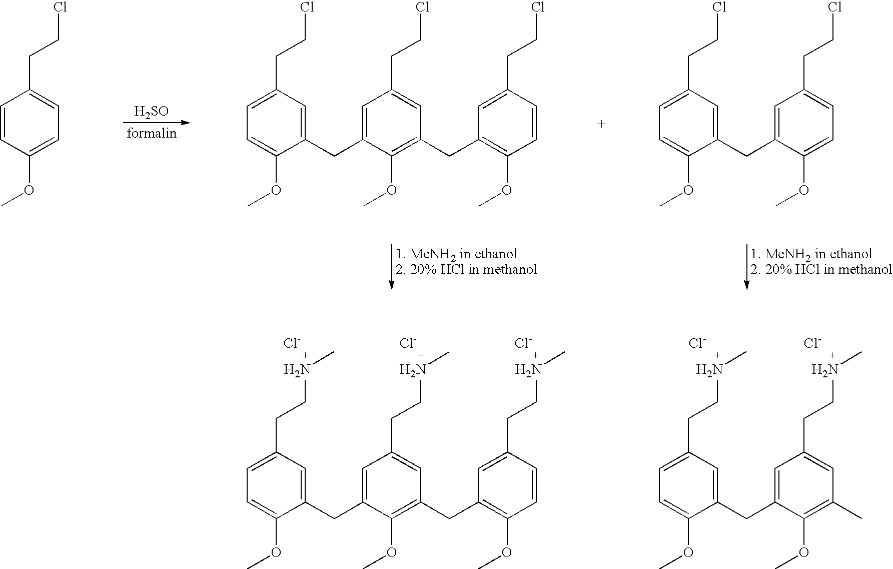

Synthesis of compounds with X as CH₂, CH₂OCH₂ as spacers, or the amino acid spacer described abovecan be achieved via:
For the CH₂ spacer, addition of suitable monomers under acidic conditions to give mixtures which are separated into the individual components (see experimental for the chloro compounds). Alternatively, an iterative procedure in solid or solution phase using a protected monomer and controlled addition of a single unit in each step (see representative scheme below).

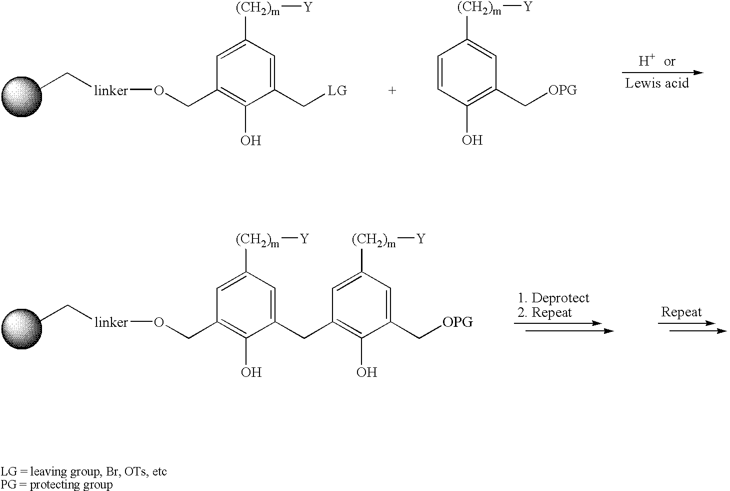

For the CH₂OCH₂, addition of monomer units in a sequential fashion through ether formation. Solution and solid phase chemistries could be utilised, and the scheme for solid phase is shown below.

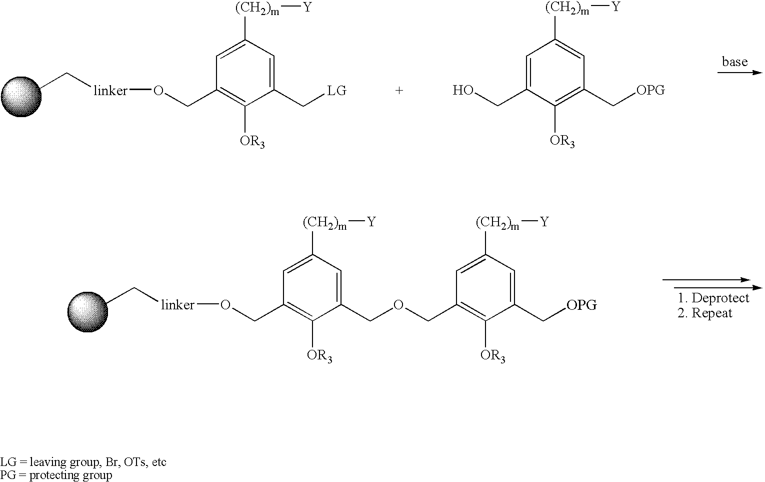

For the amino acid linker (X) described above, comprised of the amino acid below (protected Fmoc building block given in Scheme below), standard solid phase peptide assembly methodology will be used to sequentially add protected analogues of the monomer. This is a much easier means of producing the higher polymerisation states. The amino acid (in racemic or chiral form) can be prepared using several strategies, for example using Evan's methodology as outlined below,

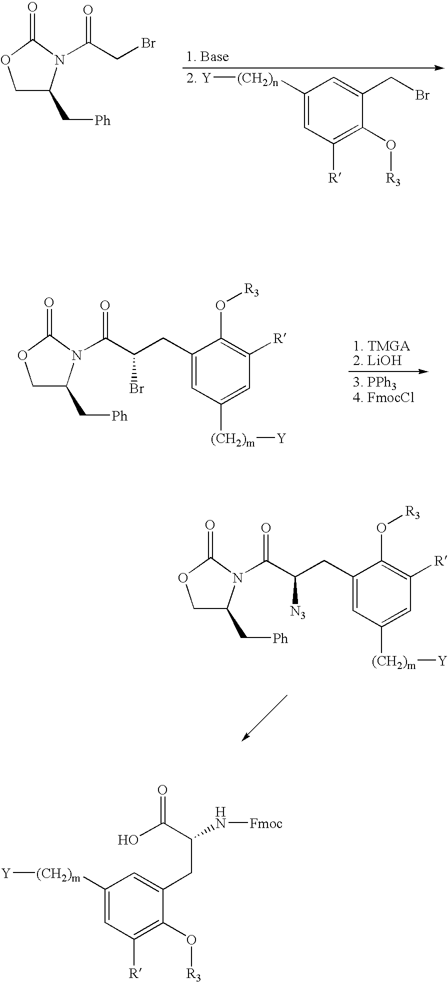

for the R-isomer (Evans and Watson 1996).

The invention will now be described with reference to the following examples, which should in no way be construed as limiting the scope of the invention. Preferred features of each aspect of the invention are as for each other aspect, mutatis mutandis.

The examples refer to the figure, which shows the results of three western blots, illustrating phosphorylation of PKB when treated with various compounds of the invention.

### EXAMPLE 1

Polymerisation of 1-(2-chloroethyl)-4-methoxybenzene

Formalin (37% formaldehyde aqueous solution) (0.8 ml) and 50% aqueous sulfuric acid (20 ml) were added to 1-(2-chloroethyl)-4-methoxybenzene (2.03 g, 12.5 mmol). The mixture was heated at reflux for 5 hours. After cooling, the aqueous layer was decanted off. Water (10 ml) and potassium hydroxide (20 ml) were added to basify the mixture to pH 11. The product was then extracted using diethyl ether (3×80 ml), dried (sodium sulfate) and the solvent was removed in vacuo. The isolated mixture (2.05 g) was separated using flash column chromatography (hexane:ethyl acetate (6:1)) to give the dimer (0.364 g, 27%) and trimer (0.147 g, 7%). The dimer could also be isolated by recrystallisation (ethyl acetate).

Dimer: 3,3′-Methylenebis[1-(2-chloroethyl)-4-methoxybenzene]

mp 102° C. (ethyl acetate);

νₘₐₓ(film)/cm⁻¹ 1436 m, 1448 m, 1463 m, 1500 s, 1605 s, 2835 w, 2860 w, 2989 w;

δ_{H}(300 MHz; CDCl₃) 2.94 (4H, t, J 7.5 Hz, 2×ArCH₂), 3.62 (4H, t, J 7.5 Hz, 2×CH₂Cl), 3.83 (6H, s, 2×OMe), 3.95 (2H, s, ArCH₂Ar), 6.81 (2H, d, J 8.2 Hz, 2×5-H), 6.88 (2H, s, 2×5-H), 7.06 (2H, d, J 8.2 Hz, 2×6-H);

δ_{C}(75 MHz; CDCl₃) 30.17 (ArCH₂Ar), 38.88 (CH₂Ar), 45.68 (CH₂Cl), 55.85 (OMe), 110.78 (CH), 127.72 (CH), 129.57, 130.22, 131.22 (CH), 157.00 (C-OMe); m/z (FAB) 352 (M⁺, 63%), 317 ([M-Cl]⁺, 15), 303 ([M-CH₂Cl]⁺, 22), 183 ([M-C₉H₉OCl]⁺, 98), 152 ([M-C₁₀H₁₂O₂Cl]⁺, 16), 117 ([M-C₁₀H₁₂O₂Cl₂]⁺, 18).

Trimer: 1-(2-Chloroethyl)4-methoxy-3,5-{bis[2-methoxy-5-(2-chloroethyl)phenyl]-methane}-benzene

νₘₐₓ(film)/cm⁻¹ 1436 m, 1461 m, 1498 m, 1609 s, 2833 w, 2909 w, 2996 m;

δ_{H}(300 MHz; CDCl₃) 2.86 (4H, t, J 7.5 Hz, 2×ArCH₂), 2.94 (2H, t, J 7.5 Hz, ArCH₂), 3.59 (6H, m, 3×CH₂Cl), 3.68 (6H, s, 2×OMe), 3.81 (3H, s, OMe), 4.09 (4H, s, 2×ArCH₂Ar), 6.73 (2H, s), 6.83 (2H, d, J 8.3 Hz, 2×3-H), 6.86 (2H, br s), 7.04 (2H, d, J 8.3 Hz, 2×2-H);

δ_{C}(300 MHz; CDCl₃) 29.93 (ArCH₂Ar), 38.83 (CH₂Ar), 39.05 (CH₂Ar), 45.51 (CH₂Cl), 45.69 (CH₂Cl), 55.88 (OMe), 61.28 (OMe) 110.84 (CH), 127.86 (CH), 129.49 (CH), 129.86, 130.35, 131.26 (CH), 133.76, 134.03 (CH), 154.0 (C-OMe), 156.87 (C-OMe);

m/z (FAB) 534 (M⁺, 20%), 365 ([M—C₉H₁₀OCl]⁺, 17), 351 ([M—C₁₀H₁₂OCl)⁺, 10), 183 ([M—C₁₉H₂₂O₂Cl₂]⁺, 100);

Benzeneethanamine-3,3′-methylenebis(4-methoxy-N-methyl) dihydrochloride

A mixture of the dimer 3,3′-methylenebis[1-(2-chloroethyl)-4-methoxybenzene] (0.21 g, 0.60 mmol) and 33% methylamine in ethanol (5.6 g, 59.8 mmol) were stirred for 12 days. The solvent was removed in vacuo to give a white solid. 1 M Hydrochloric acid (20 ml) was used to acidify the mixture to pH 1 and then the starting material was extracted using diethyl ether (3×50 ml). Then 2 M potassium hydroxide (30 ml) was added to the aqueous layer to a pH of 10. The product was extracted using diethyl ether (4×60 ml) and dried (sodium sulphate). The solvent was removed in vacuo to yield the dimer (0.18 g, 91%).

20% HCl-methanol was added to give a white solid. The solid was collected to give the hydrochloride salt (0.03 g, 13%).

νₘₐₓ(film)/cm⁻¹ 1465 m, 1504 s, 1602 w, 2419 w, 2744 m, 2942 m;

δ_{H}(300 MHz; D₂O) 2.62 (6H, s, 2×NMe), 2.85 (2H, t, J 7.5 Hz, ArCH₂), 3.17 (2H, t, J 7.5 Hz, CH₂N), 3.77 (6H, s, 2×OMe), 3.86 (2H, s, ArCH₂Ar), 6.99 (2H, d, J 6.1 Hz, 2×5-H ), 6.02 (2H, s, 2×2-H), 7.15 (2H, d, J 6.1 Hz, 2×6-H);

δ_{C}(300 MHz; D₂O) 30.16 (ArCH₂Ar), 31.16 (CH₂Ar), 33.13 (CH₃N), 50.56 (CH₂N), 56.29 (OMe), 112.6 (CH), 128.5, 129.1 (CH-6), 129.8, 131.0, 156.7 (C-OMe); m/z (FAB) 535 (M⁺, 40), 520 ([M-CH₃]⁺, 100).

Benzeneethanamine-4-methoxy-3,5-bis{(2-methoxy-5-(2-methylaminoethyl) phenyl]methyl-N-methyl trichloride^{(Katsu, Ono et al. 1984)}

A mixture of 1-(2-chloroethyl)4-methoxy-3,5-{bis[2-methoxy-5-(2-chloroethyl)phenyl]methane}benzene (0.13 g, 0.19 mmol) and 33% methylamine in ethanol (6.83 g, 56.2 mmol) were stirred for 12 days. The solvent was removed in vacuo. 1 M Hydrochloric acid (20 ml) was used to acidify the mixture to pH 1 and then the starting material was extracted using diethyl ether (3×40 ml). Then 2 M potassium hydroxide (30 ml) was added to the aqueous layer to basify the solution to pH 10. The product was extracted using diethyl ether (3×50 ml) and dried (sodium sulphate). The solvent was removed in vacuo to give a light brown viscous oil.

20% HCl-methanol was added to the oil to produce a precipitate which was removed by filteration. The salt was extracted using water. The trimer was then isolated using flash chromatography (28% ammonia: ethanol (1:1)) to give the salt (0.002 g, 2%).

δ_{H}(300 MHz; D₂O) 2.59 (3H, s, NMe), 2.68 (6H, s, 2×NMe), 2.84 (2H, t, J 7.0 Hz, ArCH₂), 2.96 (4H, t, J 7.4 Hz, 2×ArCH₂), 3.07 (2H, t, J 7.0 Hz, CH₂N), 3.21 (4H, t, J 7.4 Hz, 2×CH₂N), 3.83 (3H, s, OMe), 3.87 (6H, s, OMe), 4.07 (4H, s, ArCH₂Ar), 6.96 (2H, s, 2-H and 6-H), 7.13 (2H, s, 2×6′-H), 7.15 (2H, d, J 8.2 Hz, 2×3′-H), 7.30 (2H, d, J 8.2 Hz, 2×4′-H).

### EXAMPLE 2

### PKB Ser473 Activation Synergy Experiments

Experiments Monitoring PKB Activation

PKB is a protein downsteam effector of PI3K, and becomes phosphorylated on (residues required for its activity) in response to the activation of PI3K. Natal Calf Serum (NCS) is a stimulator of PI3K and thus subsequently results in PKB activation. Therefore the positive control used in experiments is 10% serum and a negative control used is provided with no serum at all.

Compound 48/80 is the condensation product of N-methyl-p-methoxyphenethylamine and formaldehyde and is a mixture of cationic amphiphiles of varying degrees of polymerisation. Previous experiments have shown that the compound 48/80 (Sigma) alone failed to stimulate PKB phosphorylation on Ser473 after 15 minutes in fresh serum-free media. It was further discovered that if the cells were first primed with 1% serum for 20 minutes, then compound 48/80 induced an activation of PKB higher than treatment with 1% serum alone. A synergistic effect. Pretreating the cells with compound 48/80 at a higher dose of 20-30 μg/mL has been shown to have an inhibitory effect on PKB activation.

In a typical method, NIH3T3 cells were grown in media (GibcoBRL) containing 10% NCS to near confluency in six well plates. The cells were starved using 0.5% serum for 2-3 days. The media was then removed and replaced with serum free media for 15 minutes. Subsequently, 1% NCS was added to reaction wells, and 0%, 1% and 10% NCS to control wells. After 20 minutes incubation, then compound was added, and the wells incubated for a further 15 minutes. Media was removed and sample buffer was added and the cells lysed, boiled, centrifuged.

Samples were subjected to gel electrophoresis by 10% SDS-PAGE and then Western blotted on to PVDF membrane (Biorad) according to standard protocols. Western blots were probed using primary antibodies against PKB purchased from New England Biolabs and secondary antibodies of goat anti-rabbit IgG coupled to horseradish peroxidase (Amersham). The membranes were then developed using a freshly prepared ECL solution according to standard protocols.

The monomer, dimer and trimer of compound 48/80 were received as synthesised from Macba Numbere at UCL. These were diluted into water initially, but after observed solubility problems, into DMSO (Sigma).

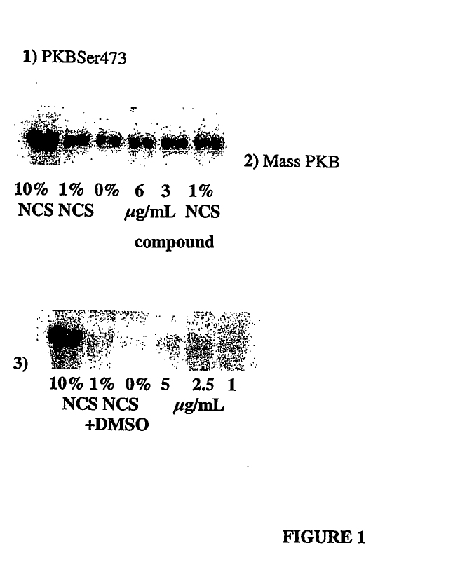

Western blot 1) in FIG. 1 illustrates the phosphorylation of PKB with respect to dose at 6 μg/mL and 3 μg/mL of trimer compound. This shows that at higher concentrations trimer compound, there is inhibition of PKB phosphorylation. At 3 μg/mL concentration there is a level of activation at a level just below that of 1% serum activation. However, at 6 μg/mL the level of activation is almost completely inhibited. Western blot 2) shows the same membrane when reprobed for all forms of PKB. These show a uniform appearance as expected for a good control, with the exception of the 10% NCS lane which is stronger and attributable to hyperphosphorylation.

Western blot 3) illustrates the reaction of PKB to the monomeric species from compound 48/80. This shows a slight activation above control levels for PKB Ser473 phosphorylation. The 1% and 0% NCS controls both contain DMSO at 2.5% concentration reflecting that for the amount of DMSO in the 5 μg/mL experiment.

### CONCLUSIONS

It appears from these studies that the trimer is an inhibiting species whereas the monomer appears to be the activating species.

### REFERENCES

Berge, S. M., L. D. Bighley, et al. (1977). “Pharmaceutical salts.” J Pharm Sci 66(1): 1-19.

Brazil, D. P., J. Park, et al. (2002). “PKB binding proteins. Getting in on the Akt.” Cell 111(3): 293-303.

Chang, F., J. T. Lee, et al. (2003). “Involvement of PI3K/Akt pathway in cell cycle progression, apoptosis, and neoplastic transformation: a target for cancer chemotherapy.” Leukemia 17(3): 590-603.

Evans, D. A. and P. A. Watson (1996). Tetrahedron Lett.: 3251-3254.

Glass, D. J. (2003). “Signalling pathways that mediate skeletal muscle hypertrophy and atrophy.” Nat Cell Biol 5(2): 87-90.

Hara, K., K. Yonezawa, et al. (1994). “1-Phosphatidylinositol 3-kinase activity is required for insulin-stimulated glucose transport but not for RAS activation in CHO cells.” Proc Natl Acad Sci U S A 91(16): 7415-9.

Hill, M., J. Feng, et al. (2002). “Identification of a Plasma Membrane Raft-Associated PKB Ser473 Kinase Activity that Is Distinct from ILK and PDK1.” Curr Biol 12(14): 1251.

Hill, M. M. and B. A. Hemmings (2002). “Inhibition of protein kinase B/Akt. implications for cancer therapy.” Pharmacol Ther 93(2-3): 243-51.

Katsu, T., H. Ono, et al. (1984). Chem. Pharm. Bull 32: 4185-4188.

Kundra, V., J. A. Escobedo, et al. (1994). “Regulation of chemotaxis by the platelet-derived growth factor receptor-beta.” Nature 367(6462): 474-6.

Matsui, T., T. Nagoshi, et al. (2003). “Akt and PI 3-kinase signaling in cardiomyocyte hypertrophy and survival.” Cell Cycle 2(3): 220-3.

Newton, A. C. (2003). “Regulation of the ABC kinases by phosphorylation: protein kinase C as a paradigm.” Biochem J 370(Pt 2): 361-71.

Parekh, D. B., W. Ziegler, et al. (2000). “Multiple pathways control protein kinase C phosphorylation.” Embo J 19(4): 496-503.

Rodriguez-Viciana, P., P. H. Warne, et al. (1994). “Phosphatidylinositol-3-OH kinase as a direct target of Ras.” Nature 370(6490): 527-32.

Sabers, C. J., M. M. Martin, et al. (1995). “Isolation of a protein target of the FKBP12-rapamycin complex in mammalian cells.” J Biol Chem 270(2): 815-22.

Schu, P. V., K. Takegawa, et al. (1993). “Phosphatidylinositol 3-kinase encoded by yeast VPS34 gene essential for protein sorting.” Science 260(5104): 88-91.

Staal, S. P. (1987). “Molecular cloning of the akt oncogene and its human homologues AKT1 and AKT2: amplification of AKT1 in a primary human gastric adenocarcinoma.” Proc Natl Acad Sci U S A 84(14): 5034-7.

Stephens, L., F. T. Cooke, et al. (1994). “Characterization of a phosphatidylinositol-specific phosphoinositide 3-kinase from mammalian cells.” Curr Biol 4(3): 203-14.

Tatton, W., D. Chen, et al. (2003). “Hypothesis for a common basis for neuroprotection in glaucoma and Alzheimer's disease: anti-apoptosis by alpha-2-adrenergic receptor activation.” Surv Ophthalmol 48 Suppl 1: S25-37.

Valius, M. and A. Kazlauskas (1993). “Phospholipase C-gamma 1 and phosphatidylinositol 3 kinase are the downstream mediators of the PDGF receptor's mitogenic signal.” Cell 73(2): 321-34.

Vanhaesebroeck, B., S. J. Leevers, et al. (2001). “Synthesis and Function of 3-Phosphorylated Inositol Lipids.” Annu Rev Biochem 70: 535-602.
Annu. Rev. Biochem. 2001. 70:535-602.SYNTHESIS AND FUNCTION OF 3-PHOSPHORYLATED INOSITOL LIPIDS

Varticovski, L., B. Druker, et al. (1989). “The colony stimulating factor-1 receptor associates with and activates phosphatidylinositol-3 kinase.” Nature 342(6250): 699-702.
Colony stimulating factor-1 (CSF-1) is a lineage-specific growth factor

Vivanco, I. and C. L. Sawyers (2002). “The phosphatidylinositol 3-Kinase AKT pathway in human cancer.” Nat Rev Cancer 2(7): 489-501.

1-14. (canceled)

15. A compound of the formula: 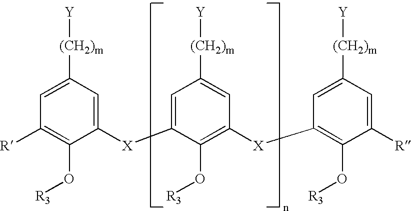
wherein Y is halogen, OH, CO₂H, CONR₁R₂, CHO or NR₁R₂;
R₁, R₂ and R₃ are each independently H or C₁₋₅alkyl;
n is 2-10 and m is 1-5;
R′ and R″ are independently H or CH₂OH;
X is CH₂, O, CH₂O or CH₂OCH₂;
and pharmaceutically acceptable salts thereof.

16. A pharmaceutical composition comprising a compound of claim 15 and one or more pharmaceutically acceptable excipients, carriers or diluents.

17. A method of altering Protein Kinase B activity in cells or tissues comprising contacting said cells or tissues with a compound of claim 15.

18. The method of claim 17 wherein the alteration of activity comprises the inhibition of Protein Kinase B phosphorylation.

19. The method of claim 18 wherein the compound is a trimer.

20. The method of claim 17 wherein the alteration of activity comprises the activation of Protein Kinase B phosphorylation.

21. A method of treating an animal having a disease or condition associated with downregulation of Protein Kinase B activity comprising administering to said animal a therapeutically effective amount of a compound of claim 15 wherein Protein Kinase B activity is activated.

22. The method of claim 21 wherein the disease or condition is a degenerative disorder and the animal is a human.

23. The method of claim 22 wherein the degenerative disorder is Alzheimers Disease.

24. The method of claim 21 wherein the disease or condition involves tissues of the heart or skeletal muscle.

25. A method of treating an animal having a disease or condition associated with upregulation of Protein Kinase B activity comprising administering to said animal a therapeutically effective amount of a compound of claim 15 wherein Protein Kinase B activity is inhibited.

26. The method of claim 25 wherein the inhibition of activity of Protein Kinase B is the result of decreased phosphorylation at Serine 473.

27. The method of claim 25 wherein the disease or condition is cancer and the animal is a human.

28. The method of claim 27 wherein a mutation of PTEN is implicated.

29. the method of claim 28 wherein the cancer is selected from the group consisting of ovarian, breast, prostate, thyroid and pancreatic.

30. The compound of claim 15 having the formula: 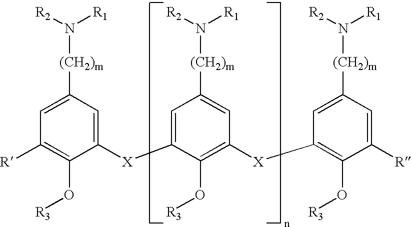
wherein R₁, R₂ and R₃ are each independently H or C₁₋₅alkyl, n is 2-10 and m is 1-5; R′ and R″ are independently H or CH₂OH and X is CH₂, O, CH₂O or CH₂OCH₂; and pharmaceutically acceptable salts thereof.

31. The compound of claim 30 wherein R₃ is Me and Y is NR₁R₂ wherein R₁ is H and R₂ is a C₁ alkyl.

32. A compound of the formula: 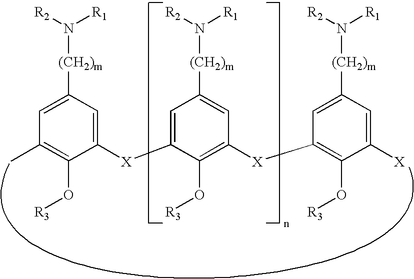
wherein R₁, R₂ and R₃ are each independently H or C₁₋₅alkyl;
n is 1-10 and m is 1-5;
X is CH₂, O, CH₂O or CH₂OCH₂;
and pharmaceutically acceptable salts thereof.

33. A pharmaceutical composition comprising a compound of claim 32 and one or more pharmaceutically acceptable excipients, carriers or diluents.

34. A method of altering Protein Kinase B activity in cells or tissues comprising contacting said cells or tissues with a compound of claim 32.

35. The method of claim 34 wherein the alteration of activity comprises the inhibition of Protein Kinase B phosphorylation.

36. The method of claim 34 wherein the alteration of activity comprises the activation of Protein Kinase B phosphorylation.

37. A method of treating an animal having a disease or condition associated with downregulation of Protein Kinase B activity comprising administering to said animal a therapeutically effective amount of a compound of claim 32 wherein Protein Kinase B activity is activated.

38. The method of claim 37 wherein the disease or condition is a degenerative disorder and the animal is a human.

39. The method of claim 38 wherein the degenerative disorder is Alzheimers Disease.

40. The method of claim 37 wherein the disease or condition involves tissues of the heart or skeletal muscle.

41. A method of treating an animal having a disease or condition associated with upregulation of Protein Kinase B activity comprising administering to said animal a therapeutically effective amount of a compound of claim 32 wherein Protein Kinase B activity is inhibited.

42. The method of claim 41 wherein the inhibition of activity of Protein Kinase B is the result of decreased phosphorylation at Serine 473.

43. The method of claim 41 wherein the disease or condition is cancer and the animal is a human.

44. The method of claim 43 wherein a mutation of PTEN is implicated.

45. the method of claim 44 wherein the cancer is selected from the group consisting of ovarian, breast, prostate, thyroid and pancreatic.

46. A compound of the formula: 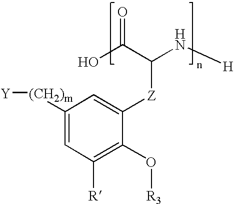
wherein Y is H, halogen, OH, CO₂H, CONR₁R₂, CHO or NR₁R₂, R₁, R₂ and R₃ are each independently H or C₁₋₅alkyl;
n is 2-12 and m is 1-5;
R′ is H or CH₂OH;
Z is (CH₂)ₙ or PEG;
and pharmaceutically acceptable salts thereof.

47. A pharmaceutical composition comprising a compound of claim 46 and one or more pharmaceutically acceptable excipients, carriers or diluents.

48. A method of altering Protein Kinase B activity in cells or tissues comprising contacting said cells or tissues with a compound of claim 46.

49. The method of claim 48 wherein the alteration of activity comprises the inhibition of Protein Kinase B phosphorylation.

50. The method of claim 49 wherein the alteration of activity comprises the activation of Protein Kinase B phosphorylation.

51. A method of treating an animal having a disease or condition associated with an alteration of Protein Kinase B activity comprising administering to said animal a therapeutically effective amount of a compound of claim 46 wherein if the disease or condition is associated with downregulation of Protein Kinase B activity, then Protein Kinase B activity is activated or if the disease or condition is associated with an upregulation of Protein Kinase B activity then Protein Kinase B activity is inhibited.

52. The method of claim 51 wherein the disease or condition is associated with downregulation of Protein Kinase B activity and is a degenerative disorder and the animal is a human.

53. The method of claim 52 wherein the degenerative disorder is Alzheimers Disease.

54. The method of claim 52 wherein the disease or condition involves tissues of the heart or skeletal muscle.

55. (canceled)

56. The method of claim 51 wherein the disease or condition is associated with upregulation of Protein Kinase B activity and the inhibition of activity of Protein Kinase B is the result of decreased phosphorylation at Serine 473.

57. The method of claim 51 wherein the disease or condition the disease or condition is associated with upregulation of Protein Kinase B activity and the disease or condition is cancer and the animal is a human.

58. The method of claim 57 wherein a mutation of PTEN is implicated.

59. The method of claim 58 wherein the cancer is selected from the group consisting of ovarian, breast, prostate, thyroid and pancreatic.

60. (canceled)
